(19) United States
(12) Patent Application Publication
(10) Pub. No.: US 2020/0384044 A1
(43) Pub. Date: Dec. 10, 2020

(54) METHOD OF DETECTING FUNCTIONAL GASTROINTESTINAL DISORDER BY MEASURING GASTRIC FLORA OCCUPANCY, AND GASTRIC FLORA IMPROVING AGENT

(71) Applicants: MEIJI CO., LTD., Chuo-ku, Tokyo (JP); TOKAI UNIVERSITY EDUCATIONAL SYSTEM, Shibuya-ku, Tokyo (JP)

(72) Inventors: Yasuhiro Koga, Isehara-shi, Kanagawa (JP); Hirohiko Nakae, Isehara-shi, Kanagawa (JP); Toshihiro Ohtsu, Odawara-shi, Kanagawa (JP)

(73) Assignees: MEIJI CO., LTD., Chuo-ku, Tokyo (JP); TOKAI UNIVERSITY EDUCATIONAL SYSTEM, Shibuya-ku, Tokyo (JP)

(21) Appl. No.: 16/473,723

(22) PCT Filed: Dec. 26, 2017
(86) PCT No.: PCT/JP2017/046512

§ 371 Date: Jun. 26, 2019

(30) Foreign Application Priority Data
Dec. 27, 2016 (JP) 2016-254357

(51) Int. Cl. A61K 35/747; C12Q 1/689; A61P 1/14

(52) U.S. Cl. CPC A61K 35/747; A61P 1/14; C12Q 1/689

(57) ABSTRACT

Provided is a method of detecting presence or absence of a functional gastrointestinal disorder by measuring bacteria occupancy within a human stomach.

### TECHNICAL FIELD

The present invention relates to a method of detecting a functional gastrointestinal disorder by measuring bacteria occupancy within a human stomach by gastric flora analysis, and a gastric flora improving agent which is found according to this detection method.

Specifically, the present invention is based on a finding in which development of the functional gastrointestinal disorder, particularly, the functional dyspepsia is related to bacteria occupancy within the human stomach, particularly, to the fact that Bacteroidetes occupancy is higher than Proteobacteria occupancy within the human stomach and/or that Acidobacteria have no occupation within the human stomach, and is based on a finding of the method of detecting the functional gastrointestinal disorder and the gastric flora improving agent.

### BACKGROUND ART

So far, even with progress of diagnostic endoscopy, there have been many cases of findings in which symptoms cannot be elucidated for complaints of upper gastrointestinal symptoms such as upper abdominal pain or discomfort, postprandial heavy stomach feeling, upper abdominal bloating, nausea, vomiting, epigastric pain and epigastric burning. A state where, even with presence of such complaints of gastrointestinal symptoms, no organic disease can be found by general examination including endoscopy and therefore no findings for elucidating the symptoms can be obtained is referred to as FD (functional dyspepsia, including indefinite complaints of upper abdomen, postprandial distress, epigastric pain, or functional gastroenteropathy).

In Japan, the functional dyspepsia (FD) has been approved as a disease name for national insurance billing purposes since 2013. The disease is judged to be FD according to Evidence-based Clinical Practice Guidelines for Functional Dyspepsia, 2014 (April 2014, Nankodo Co., Ltd., Non-Patent Document 1) edited by the Japanese Society of Gastroenterology.

Moreover, these symptoms of FD are not shown in the organic disease, and therefore there have been problems in which the symptoms are overlooked or misdiagnosed. More specifically, there have been adverse effects in which, while the symptom is not diagnosed under a clear disease name even with such discomfort, quality of life (QOL) of a person who has a functional gastrointestinal disorder decreases or treatment is delayed.

On the other hand, as a method of improving these symptoms, administration of serotonin or a drug for releasing nitrogen monoxide is known. However, administration of such a drug is accompanied by a side effect, and therefore it has been expected to prevent or improve the functional gastrointestinal disorder by a method without being accompanied by the side effect.

In order to prevent or improve the functional gastrointestinal disorder by the method without being accompanied by the side effect, various proposals have been made so far.

For example, Patent Document 1 and Patent Document 2 describe that the functional gastrointestinal disorder has been improved by administering glutamic acid, 5′-nucleotide or the like (claim 1 in each document).

Patent Document 3 describes a preventing/improving agent for functional gastrointestinal disorders, which comprises glutamic acid, arginine and the like as active ingredients. Patent Document 3 describes that this preventing/improving agent can be produced conveniently at a low cost, is highly safe, and is effective particularly for upper gastrointestinal functional disorders such as functional dyspepsia (FD) including abdominal pain, heavy stomach feeling and burning pain and gastroesophageal reflux disease (GERD) (abstract in the document).

Moreover, as a technology of preventing or improving the functional gastrointestinal disorder by using lactic acid bacteria, Patent Document 4 describes Bifidobacterium bifidum (hereinafter, referred to as Bifidobacterium in several cases) that has an effect of eradicating Helicobacter pylori (hereinafter, referred to as H. pylori in several cases) and exhibits high persistence even under aerobic conditions as in fermented milk beverage, and describes that a gastric indefinite complaint syndrome has been improved by taking this fermented milk beverage containing Bifidobacterium (paragraphs 0011 and 0096 in the document).

Patent Document 5 describes that a Lactobacillus gasseri MCC 1183 strain exhibits an effect of eradicating H. pylori, and describes anti-inflammatory agents and anti-ulcer agents for use in preventing or treating gastritis applying this finding, as well as foods and drinks suitable for relieving heavy stomach feeling (paragraphs 0023 and 0052 in the document).

Patent Document 6 describes a gastrointestinal hyperfunction agent containing lactic acid bacteria of the genus Lactobacillus, such as Lactobacillus acidophilus (Lactobacillus gasser!), lactic acid bacteria of the genus Streptococcus, such as Streptococcus faecalis, and aloe (claims 1 and 2, and paragraph 0012 in the document), and describes that gastrointestinal hyperfunction also includes improvement of heavy stomach feeling and abdominal bloating (paragraph 0043 in the document).

More specifically, a technology of eradicating H. pylori to improve the functional gastrointestinal disorder by the fermented milk beverage containing lactic acid bacteria such as Bifidobacterium has been already known. Moreover, eradicating H. pylori by using Lactobacillus gasseri MCC 1183 strain to improve the heavy stomach feeling has also been already proposed. Further, improving the heavy stomach feeling and the abdominal bloating by the gastrointestinal hyperfunction agent containing lactic acid bacteria of the genus Lactobacillus, lactic acid bacteria of the genus Streptococcus and aloe has also been already proposed.

### RELATED ART DOCUMENTS

### Patent Documents

Patent Document 1: W02006/030980
Patent Document 2: Japanese Patent No. 5067145
Patent Document 3: W02009/113594
Patent Document 4: Japanese Patent No. 4881304
Patent Document 5: Japanese Patent No. 5300772
Patent Document 6: JP-A-2012-126700
Patent Document 7: Japanese Patent No. 4509250
Patent Document 8: WO2015/129281

### Non-Patent Document

Non-Patent Document 1: Evidence-based Clinical Practice Guidelines for Functional Dyspepsia, 2014 (Nankodo Co., Ltd., April 2011) edited by the Japanese Society of Gastroenterology

### SUMMARY OF THE INVENTION

In a functional gastrointestinal disorder, particularly, functional dyspepsia, these symptoms are not shown in an organic disease, and therefore there has been a problem in which the symptoms are overlooked or misdiagnosed.

On the other hand, as described in Patent Document 7, the present applicant has a patent right on a medicament for eradicating H. pylori and/or for preventing infection with H. pylori, which contains as an active ingredient Lactobacillus gasseri OLL2716 strain that is lactic acid bacteria having high capability of eradicating H. pylori, Patent Document 7 describes that the medicament is used as an anti-gastritis agent or an anti-ulcer agent (claims 1 and 3 in the document).

Moreover, as described in Patent Document 8, the present applicant has found that Lactobacillus gassed OO2716 strain is effective in preventing and/or improving the functional gastrointestinal disorder.

However, it has been unclear as to by what mechanism Lactobacillus gasseri OL2716 strain is effective as the agent for preventing and/or improving the functional gastrointestinal disorder.

Accordingly, an objective of the present invention is to provide a new method of detecting presence or absence of the functional gastrointestinal disorder, particularly, the functional dyspepsia.

The present inventors have diligently conducted research, and have found by gastric flora analysis that bacteria occupancy within a human stomach, particularly, the fact that Bacteroidetes occupancy is higher than Proteobacteria occupancy within the human stomach and/or that Acidobacteria have no occupation within the human stomach, is related to development of the functional gastrointestinal disorder, particularly, the functional dyspepsia, and thus have completed the present invention.

According to research by the present inventors, it has been found that Lactobacillus gasseri OLL2716 strain reduces the Bacteroidetes occupancy relative to the Protecbacteria occupancy within a stomach for persons negative for H. pylori, arid that the strain increases Acidobacteria occupancy within the stomach.

Moreover, at this case, the present inventors have conducted research on an effect of reducing the Bacteroidetes occupancy relative to the Proteobacteria occupancy within the stomach and on an effect of increasing the Acidobacteria occupancy within the stomach by Lactobacillus gasseri OLL2716 strain, and as a result, the present inventors have found that, surprisingly, there is no relationship with an effect of Lactobacillus gasseri OLL2716 strain to eradicate H. Pylori.

The present invention provides the following items.

1. A method of detecting presence or absence of a functional gastrointestinal disorder, comprising measuring bacteria occupancy within a human stomach.

2. The method according to 1, wherein the bacteria occupancy within the human stomach is measured on gastric juice collected from inside of the human stomach.

3. The method according to 1 or 2, comprising measuring that Bacteroidetes occupancy is higher than Proteobacteria occupancy within a human stomach and/or that Acidobacteria have no occupation within the human stomach.

4. The method according to any one of 1 to 3, wherein the functional gastrointestinal disorder is functional dyspepsia.

5. The method according to any one of 1 to 4, wherein the bacteria occupancy is measured by using a next-generation sequencer.

6. A gastric flora improving agent, containing lactic acid bacteria as an active ingredient, wherein the gastric flora improving agent reduces Bacteroidetes occupancy relative to Proteobacteria occupancy within a stomach, and/or the gastric flora improving agent increases Acidobacteria occupancy within the stomach.

7. The gastric flora improving accent according to 6, wherein the lactic acid bacteria are lactic acid bacteria of a genus Lactobacillus.

8. The gastric flora improving agent according to 6 or 7, wherein the lactic acid bacteria are Lactobacillus gasseri OLL2716 (PERM BP-6999).

9. The gastric fora improving agent according to any one of 6 to 8, wherein a dose of the number of the lactic acid bacteria to a human per day is 2×10⁷ to 5×10¹⁰.

10. The gastric flora improving agent according to any one of 6 to 9, wherein, when a culture of the lactic acid bacteria contains 10⁷ or more of lactic acid bacteria per gram of the culture, a dose of the culture of the lactic acid bacteria to a human per day is 5 to 1000 g.

11, The gastric flora improving agent according to any one of 6 to 10, wherein the gastric flora improving agent produces a gastric flora improving effect within 4 weeks after intake of the gastric flora improving agent.

12. The gastric flora improving agent according to any one of 6 to 11, wherein the gastric flora improving agent is for use in persons negative for Helicobacter pylori and persons positive for Helicobacter pylori.

13. The gastric flora improving agent according to any one of 6 to 12, wherein the gastric flora improving agent is for use in the persons negative for Helicobacter pylori.

14. The gastric flora improving agent according to any one of 6 to 13, wherein the gastric flora improvement is measured by using a next-generation sequencer.

The present invention can provide a new method of detecting presence or absence of the functional gastrointestinal disorder, particularly, the functional dyspepsia.

A method of measuring bacteria occupancy within a human stomach by the gastric flora analysis according to an embodiment of the present invention is preferably performed by using a next-generation sequencer. The gastric flora analysis by using the next-generation sequencer can be conducted by the method described in Examples. In addition thereto, the gastric flora analysis may be conducted by any method according to which a gastric flora can be identified and quantitatively determined, such as a POR method and a cultural method.

In the present invention, the term “bacteria occupancy within the stomach” means a proportion of specific bacteria in a total number of bacteria within the stomach.

The gastric flora improving agent according to the embodiment of the present invention is for use in the persons positive for Helicobacter pylon and the persons negative for Helicobacter pylon, and formed into a composition containing lactic acid bacteria as the active ingredient.

As the lactic acid bacteria, lactic acid bacteria of the genus Lactobacillus are preferably used, and as the lactic acid bacteria of the genus Lactobacillus, Lactobacillus gasseri OLL2716 (FERM BP-6999) are more preferably used.

Further, the gastric flora improving agent according to the embodiment of the present invention is preferably for use in the persons negative for H. pylon.

Further, in the gastric flora improving agent according to the embodiment of the present invention, a dose of the number of the lactic acid bacteria to a human per day is preferably adjusted to 2×10⁷ to 5×10¹⁰, and when a culture of the lactic acid bacteria contains 10⁷ or more of the lactic acid bacteria per gram of the culture, the dose of the culture of the lactic acid bacteria to the human per day is preferably adjusted to 5 to 1000 g.

Moreover, the gastric flora improving agent according to the embodiment of the present invention has fast-acting properties, and in the fast-acting properties, the gastric flora improving agent produces a functional gastrointestinal disorder improving effect within 4 weeks after intake of the gastric flora improving agent.

Further, the gastric flora improving agent according to the embodiment of the present invention is specifically an agent for reducing Bactercidetes occupancy relative to Proteobacteria occupancy within the stomach or an agent for increasing Acidobacteria occupancy within the stomach.

Moreover, the gastric flora improving agent according to the embodiment of the present invention is preferably also formed into the composition to be provided as foods and drinks, dietary supplements, foods with health claims, and functional foods such as supplements.

According to the present invention, presence or absence of the functional gastrointestinal disorder, particularly, the functional dyspepsia can be detected by the gastric flora analysis by measuring that the Bacteroidetes occupancy is higher than the Proteobacteria occupancy within the human stomach and/or that Acidobacteria have no occupation within the human stomach.

Moreover, according to the gastric flora :proving agent of the present invention, an effect of reducing the Bacteroidetes occupancy relative to the Proteobacteria occupancy within the human stomach and/or an effect of increasing the Acidobacteria occupancy within the human stomach can be provided on both the persons positive for H. pylori and the persons negative for H. pylori.

### Description of the Embodiments

Hereinafter, preferred embodiments of the present invention will be described in detail.

A method of detecting presence or absence of a functional gastrointestinal disorder according to the embodiment of the present invention is characterized by measuring bacteria occupancy within a human stomach, and specifically by measuring that Bacteroidetes occupancy is higher than Proteobacteria occupancy and/or that Acidobacteria have no occupation.

The term “functional gastrointestinal disorder” in the embodiment of the present invention means clinical conditions in which an organic disease such as a peptic ulcer or a cancer symptom is not recognized, but such indefinite complaints of upper abdomen continue as dyspepsia, heavy stomach feeling, abdominal bloating, nausea, vomiting, upper abdominal pain, anorexia, and bowel movement disturbance, and means symptoms in which reproducible gastrointestinal symptoms to decrease QOL of a patient are recognized, even if the organic disease of a gastrointestinal tract is not found. Such a functional gastrointestinal disorder is a disease that has been so far diagnosed as chronic gastritis or gastritis, and is characterized by showing the symptoms such as abdominal pain, heavy stomach feeling and heartburn. Moreover, in such a functional gastrointestinal disorder, no organic disease of the gastrointestinal tract is found, and therefore as a cause thereof, there are several hypotheses such as abnormal transmission in a nervous system due to stress or the like, presence of minor inflammation to a degree of being unable to be detected by endoscopy, gastrointestinal hypomotility, or the like. However, the cause has not been clearly elucidated yet.

In addition, the term “gastrointestinal tract” means a series of hollow organs from an oral cavity to an anus that are engaged in digestion, Specific examples thereof include pharynx, esophagus, stomach, small intestine (duodenum, jejunum and ileum), and large intestine.

Moreover, the expression “detecting presence or absence of the functional gastrointestinal disorder” in the embodiment of the present invention means detecting of either a state of presence of the functional gastrointestinal disorder or a state of absence of the functional gastrointestinal disorder.

The term “functional dyspepsia” in the embodiment of the present invention means a disease in which, even though various symptoms such as stomach pain and heavy stomach feeling continue chronically, organic disturbance such as stomach ulcer, duodenal ulcer and stomach cancer cannot be found even if endoscopy or the like is performed, in which specific examples of main symptoms thereof include “postprandial heavy stomach feeling to be unendurable”, “a feeling of inability of eating any more foods because the stomach seems to be filled with foods immediately after starting meals (early feeling of satiety)”, “pain of a pit of the stomach (epigastric pain)”, and “burning of a pit of the stomach (epigastric burning)”,

The term “bacteria” in the embodiment of the present invention means bacteria academically systematically classified into Phylum, Class, Order, Family, Genus and Species.

As the Phylum of the bacteria in the embodiment of the present invention, as one example, specific examples thereof include Firmicutes, Bacteroidetes, Proteobacteria, Fusobacteria and Acidobacteria .

The total number of bacteria within the stomach and the bacteria occupancy within the stomach in the embodiment of the present invention are measured by a PCR amplified product of a 16S ribosomal RNA (16SrRNA) gene and next-generation sequencing. In PCR amplification of the 16S ribosomal RNA (16SrRNA) gene, it is also important to efficiently isolate a genome DNA of the bacteria from a specimen collected from a subject in order for a data group of a nucleotide sequence of the 16SrRNA gene of flora to reflect a flora structure of flora within the stomach of the subject, which can be appropriately performed by the technique well known to those skilled in the art. Specifically, the genome DNA of the bacteria contained in the flora is isolated without causing variation in isolation efficiency depending on bacterial strains, and the resulting isolated material is used as a sample for determining the nucleotide sequence. As used also in Examples mentioned later, various conventionally-known methods are known as such techniques, and therefore the total number and the occupancy can be measured by utilizing or according to the techniques.

The total number of bacteria within the stomach and the bacteria occupancy within the stomach in the embodiment of the present invention are measured by the PCR amplified product of the 16S ribosomal RNA (16SrRNA) gene and the next-generation sequencing. As used also in Examples mentioned later, various conventionally-known techniques are known, and therefore the next-generation sequencing can be performed by using the next-generation sequencer by utilizing or according to the techniques. In the next-generation sequencing, the bacteria can be accurately identified and quantitatively determined, as compared with a T-RFLP method in which accurate identification and quantitative determination of the bacteria are difficult by overlapped peaks in electrophoresis. In the next-generation sequencing, the total number of bacteria within the stomach can be measured by performing genome analysis of all 16SrRNA by using the next-generation sequencer.

In the embodiment of the present invention, the expression “the Bacteroidetes occupancy is “higher” than the Proteobacteria occupancy” means that a quotient obtained by dividing “Bacteroidetes occupancy” by “Proteobacteria occupancy” is more than 1. The quotient obtained by dividing “Bacteroidetes occupancy” by “Proteobacteria occupancy” is for example 1.01 or more, preferably 1.1 or more, more preferably 1.3 or more, and further preferably 1.6 or more.

In the embodiment of the present invention, the expression “Acidobacteria have “no occupation” means that a level of Acidobacteria occupancy is less than 0.1%, or is equal to or less than a detection limit.

A gastric flora improving agent according to the embodiment of the present invention is characterized in that the gastric flora improving agent is for use in persons positive for H. pylori and persons negative for H. pylori, and contains lactic acid bacteria as an active ingredient.

The embodiment of the present invention can provide the gastric flora improving agent containing, as the active ingredient, the lactic acid bacteria generally having an eating habit and having almost no side effect. For example, the embodiment of the present invention can provide the gastric flora improving agent even for the persons negative for H. pylori.

The lactic acid bacteria are generally used for a fermented food such as yogurt, cheese, butter and pickles, and some foods have familiar taste, and therefore the lactic acid bacteria can be easily taken.

In the lactic acid bacteria in the embodiment of the present invention, the genus, the species an origin thereof or the like is arbitrary as long as the lactic acid bacteria assimilate sugar to produce lactic acid. Above all, the lactic acid bacteria of the genus Lactobacillus, particularly, Lactobacillus gasseri are preferable, and Lactobacillus gasseri OLL2716 (FERM BP-6999) can be preferably used.

Moreover, in the embodiment of the present invention, as an effective dose (intake), the number of the lactic acid bacteria as the active ingredient is incorporated into the gastric flora improving agent so as to be taken, to the human per day, preferably in 2×10⁷ to 5×10¹⁰, more preferably in 5×10⁷ to 5×10¹⁰, further preferably in 1×10⁸ to 5×10¹⁰, still further preferably in 5×10⁸ to 5×10¹⁰, and still further preferably in 5×10⁸ to 2×10¹⁰.

The reason is that, if the number of the lactic acid bacteria is incorporated into the gastric flora improving agent so as to be taken in the number less than 2×10⁷, an effect of preventing and/or improving a human functional gastrointestinal disorder becomes hard to obtain, and even if the number of the lactic acid bacteria is incorporated into the gastric flora improving agent so as to be taken in the number more than 5×10¹⁰, no significant change in the effect is observed.

Moreover, in the embodiment of the present invention, as the effective dose (intake), when 10⁷ or more of the lactic acid bacteria, per gram of the culture of the lactic acid bacteria, are incorporated thereinto, the culture of the lactic acid bacteria is preferably incorporated into the gastric flora improving agent so as to be taken, to the human per day, preferably in 5 to 1000 g, more preferably in 10 to 1000 g, further preferably in 50 to 500 g, still further preferably in 70 to 300 g, still further preferably in 70 to 250 g, and particularly preferably in 80 to 200 g. Here, in the embodiment of the present invention, the effective dose (intake) thereof may be taken, to the human per day, once or a plurality of times of twice or more times.

The number of the lactic acid bacteria per gram of the culture of the lactic acid bacteria to be incorporated thereinto only needs to be 10⁷ or more, and may be 10⁷, 10⁸, 10⁹ or the like. If the number of the lactic acid bacteria per gram of the culture of the lactic acid bacteria to be incorporated thereinto is increased, the effective dose of the culture of the lactic acid bacteria can be reduced while the effective dose of the number of the lactic acid bacteria is incorporated into the elastic flora improving agent, and a comparable human gastric flora improvement effect can be obtained by taking a smaller amount of the culture of the lactic acid bacteria.

The culture of the lactic acid bacteria in the embodiment of the present invention can be obtained by culturing (growing) the lactic acid bacteria with a publicly-known culture medium ingredient, Moreover, the number of the lactic acid bacteria per unit weight of a culture solution can be increased by centrifuging the thus obtained culture solution of the lactic acid bacteria, or the like. The lactic acid bacteria in the embodiment of the present invention may be in a state immediately after culturing (growing) the lactic acid bacteria, in a frozen state in which the lactic acid bacteria are mixed with a cryoprotective agent or the like, or in a frozen-dried state. Moreover, the lactic acid bacteria in the embodiment of the present invention may be viable or non-viable, but is preferably viable,

Moreover, a commercially available product into which the lactic acid bacteria in the embodiment of the present invention are incorporated may be used for convenience. For example, in the case of Lactobacillus gasseri OLL2716 (FERM BP-6999), “MEIJI PROBIO YOGURT LG21” sold by Meiji Co., Ltd. can be used for convenience. The commercial item may be directly taken or may be further processed. When the lactic acid bacteria in the embodiment of the present invention and other ingestible ingredients are taken together, other ingestible ingredients are not limited, but a lactic ingredient is preferably used, for example. The term “lactic ingredient” means milk itself, or a composition containing a milk ingredient prepared by processing milk. Specific examples thereof include all ingredients containing the milk ingredient, such as raw milk (normal liquid milk or the like), recombined milk (powdered milk, cream, butter or the like), fermented milk (yogurt, cheese or the like), and a milk preparation (whey, casein, lactose, whey mineral, permeate or the like), in which the origin or a form thereof is not particularly limited.

Moreover, the gastric flora improving agent according to the embodiment of the present invention is characterized by having fast-acting properties particularly to produce the gastric flora improving effect within 4 weeks, which does not obviously limit continuously taking of the gastric flora improving agent according to the embodiment of the present invention for a period longer than 4 weeks. The gastric flora improving agent according to the embodiment of the present invention is preferably taken continuously for 4 weeks or longer, more preferably for 8 weeks or longer, further preferably for 12 weeks or longer, still further preferably for 16 weeks or longer, and still further preferably for 20 weeks or longer.

Further, in the gastric flora improving agent according to the embodiment of the present invention, an intake method and an intake frequency are not particularly limited. In Examples described later, as one example, the gastric flora improving agent is taken every day. Moreover, in the embodiment described above, the preferable number of the lactic acid bacteria to be taken per day is described, but such description does not mean that the gastric flora improving effect according to the embodiment of the present invention is recognized only when the gastric flora improving agent is surely taken every day. As long as the effect is recognized, the intake frequency can be appropriately adjusted to once every other day, once every three days, once every four days, once every five days, once every seven days (once a week), once every ten days, once a month, or the like, for example.

The gastric flora improving agent according to the embodiment of the present invention can be packed into the form of unit package per serving, and can also be packed into the form including the number of active lactic acid bacteria in the unit package.

For example, the lactic acid bacteria being the active ingredient are incorporated thereinto so as to be taken in the unit package preferably in 2×10⁷ to 5×10¹⁰, more preferably in 5×10⁷ to 5×10¹⁰, further preferably in 1×10⁸ to 5×10¹⁰, still further preferably in 5×10⁸ to 5×10¹⁰, and still further preferably in 5×10⁸ to 2×10¹⁰,

Moreover, for example, when the culture of the lactic acid bacteria contains 10⁷ or more of the lactic acid bacteria per gram of the culture, preferably in 5 to 1000 g, more preferably in 10 to 1000 g, further preferably in 50 to 500 g, still further preferably in 70 to 300 g, still further preferably in 70 to 250 g, and particularly preferably from 80 to 200 g of the culture of the lactic acid bacteria is contained as the active ingredient in the unit package.

When the gastric flora improving agent according to the embodiment of the present invention is packaged in the unit package, a publicly-known package can be used. Specific examples thereof include a package made of paper, plastics, glass, nylon, stainless steel, aluminum, iron, copper, silver, and bamboo, but are not particularly limited thereto. However, in view that the lactic acid bacteria are also facultative anaerobic bacteria, the package is preferably applied in the form of causing no contact with air or oxygen. For example, a process for eliminating possibility of contact with oxygen is preferably provided in a process of manufacturing or a process of packaging the gastric flora improving agent according to the embodiment of the present invention, and a packaging material in which no oxygen is permeated through an inside of the package in storage after packaging is preferably selected.

In the embodiment of the present invention, a method of taking the gastric flora improving agent is not particularly limited, and all publicly-known forms of taking the gastric flora improving agent, such as oral intake, tube feeding, enteral feeding, vascular injection, an ointment and a suppository can be used. In particular, oral intake can be preferably used,

In the embodiment of the present invention, a temperature of the gastric flora improving agent in taking the gastric flora improving agent is adjusted preferably to −30 to 50° C., more preferably to −20 to 45° C., further preferably to 0 to 45° C., still further preferably to 0 to 30° C., still further preferably to 0 to 20° C., and particularly preferably to 0 to 10° C.,

In the embodiment of the present invention, as an ingredient other than the lactic acid bacteria, other ingestible ingredients, various additives, foods and drinks, raw materials of pharmaceutical products or the like may be incorporated into the gastric flora improving agent.

Moreover, the gastric flora improving agent according to the embodiment of the present invention is also preferably formed into the composition to be provided as the foods and drinks, the dietary supplements, the foods with health claims, and the functional foods such as the supplements. Here, the term “functional foods” means foods having a physiological function being the third function of the functions of the food.

Then, the term “dietary supplements” means health foods certified by Japan Health & Nutrition Food Association (JHFA), and the term “foods with health claims” means foods for specified health uses and foods with nutrient function claims under the jurisdiction of Japan Consumer Affair Agency. Then, the foods and drinks also include foods and drinks that do not fall under the functional foods.

Further, the gastric flora improving agent according to the embodiment of the present invention can be processed into the form of beverage, yogurt, cheese, dessert or the like, and simultaneously processed into a product suitable for the form thereof in taste and/or physical properties thereof, or the like in order to be able to take the gastric flora improving agent continuously and without losing interest therein.

The expression “reduces the Bacteroides occupancy relative to the Proteobacteria occupancy within the stomach” in the embodiment of the present invention means reduction of the quotient (Bacteroides occupancy/Proteobacteria occupancy) obtained by dividing “Bacteroides occupancy” by “Proteobacteria occupancy”. When the quotient obtained by dividing “Bacteroides occupancy” by “Proteobacteria occupancy” is 1 or more, this quotient is reduced preferably by 0.1 or more, more preferably by 0.5 or more, and further preferably by 1 or more, and this quotient obtained by dividing “Bacteroides occupancy” by “Proteobacteria occupancy” is most preferably reduced until the quotient reaches 1 or less.

In the embodiment of the present invention, the Acidobacteria occupancy is increased preferably by 0.1% or more, more preferably by 0.5% or more, and further preferably by 1% or more. Moreover, an upper limit of the occupancy to be increased is not limited, but the occupancy is increased preferably by 50% or less, more preferably by 45% or less, and further preferably by 40% or less, for example.

### EXAMPLES

Hereinafter, a test conducted for confirming an effect of an embodiment of the present invention will be described in detail, but the present invention is not limited to the following structure.

### Example 1

Gastric juice of each of functional dyspepsia patients and healthy persons was collected, and flora was analyzed by using a next-generation sequencer.

Specifically, 24 patients having functional gastrointestinal disorders (a functional dyspepsia patient group, an ED group), who had no organic disease and were negative for H. pylori, were applied as targets. Moreover, as a control, 21 healthy persons (a healthy control group, an HC group) who had no organic disease and were negative for H. pylori were applied as targets.

An analysis method by using the next-generation sequencer is as described below.

### PCR Amplified Product of 16SrRNA Gene and Next-Generation Sequencing

The gastric juice was collected from a stomach of each subject, and DNA of bacteria was isolated from the gastric juice by using a DNA isolation kit “UltraClean® Soil DNA Isolation Kit” (Mo Bio Laboratories, Carlsbad, Calif., USA) and according to an instruction manual of the present kit. V3-V4 hypervariable regions of 16SrRNA were amplified by PCR using a 341f primer and an R806 primer. The PCR was performed according to the method reported by Takahashi S, et al. (Takahashi S, et al. “Development of a prokaryotic universal primer for simultaneous analysis of Bacteria and Archaea using next-generation sequencing”, PLoS ONE (9) 8 (2014): e105592.). Specifically, 25 μL of a reaction mixture obtained by adding MightyAmp for Real Time (SYBR Plus) (Takara, Kyoto, Japan) and 0.25 μM of each primer to 10 ng of a DNA gene was provided for the PCR. In the PCR, initial denaturation was performed at 98° C. for 2 minutes, and then annealing for 15 seconds, started at 65° C. and ended at 55° C., was performed 35 cycles, and extension reaction was performed at 68° C. for 30 seconds. An annealing temperature was decreased by 1° C. every cycle until the temperature reached 55° C. set as a holding cycle. A PCR product was purified by MultiScreen PCR μ96 Filter Plate (Merck Millipoa, USA).

Sequencing was performed by using Illumine Miseq sequencing system (Illumina, San Diego, Calif., USA) and Miseq Reagent Kit version3 chemistry and according to paired-end at an operation cycle of 2×300 bases. As a quality filtering method of the sequencing, only reads in which 99% or more of sequences satisfied a quality value score of 20 or more were extracted and used for the next analysis.

### OTU (Operational Taxonomic Unit) Analysis Based on 16SrDNA

About 30,000 of high-quality reads per sample were arbitrarily selected from the reads that passed a quality filter, and the selected reads were classified into OTU by using Metagenome@KIN analysis software (World Fusion, Tokyo, Japan) and TechnoSuruga Laboratory Microbial Identification Database DB-BA 9.0 (TechnoSuruga Laboratory, Shizuoka-city, Japan), and setting a sequence identity threshold to 97% according to the method of Hisada et al. (Hisada T, et al., “Inter- and intra-individual variations in seasonal and daily stabilities of the human gut microbiota in Japanese”, Arch Microbial, 197, pp.919-34 (2015)).

A bacteria composition was analyzed by narrowing the bacteria down to the genus in an amount more than 0.1% of total gastric juice bacteria, in which a total reached approximately 85% in the present Example. Hierarchical cluster analysis was conducted by using GeneMaths software (Applied Maths, Brussels, Belgium) based on an unweighted pair group method (UPGMA), a whole flora structure and a frequency identified into the genus for each sample were analyzed.

As a result, averages of main bacteria occupancy (bacteria composition proportion, %) in the gastric juice for the ED group and the HC group are as shown in Table 1.

TABLE 1

| | |
|---|---|
| Name of bacteria | Average of occupancy in gastric juice (%) |

| (Phylum) | FD group | HC group |
|---|---|---|
| Firmicutes | 36.0 | 31.6 |
| Bacteroidetes | 24.2 | 13.5 |
| Proteobacteria | 14.9 | 29.5 |
| Fusobacteria | 4.0 | 4.9 |
| Acidobacteria | 0.2 | 3.8 |

In the FD group, the number of persons in which Bacteroidetes occupancy was higher than Proteobacteria occupancy was 19, and the number of persons in which the Bacteroidetes occupancy was lower than the Proteobacteria occupancy was 5. Moreover, in the HC group, the number of persons in which the Bacteroidetes occupancy was higher than the Proteobacteria occupancy was 2, and the number of persons in which the Bacteroidetes occupancy was lower than the Proteobacteria occupancy was 19. When the FD group was compared with the HC group, a statistical significant difference was observed. As a p value at this time, p was smaller than 0.01 (p<0.01).

Further, a relationship between the Bacteroidetes occupancy being higher than the Proteobacteria occupancy, and FD was examined on the occupancy (%) of the bacteria in the gastric juice of each of the subjects, sensitivity was 79.2% and specificity was 90.4%. More specifically, the relationship between the Bacteroidetes occupancy being higher than the Proteobacteria occupancy, and FD was shown.

Moreover, such results were obtained that Acidobacteria occupancy in the gastric juice in the FD group was significantly low (no occupation) as compared with the occupancy thereof in the HC group. In the FD group, the number of persons in which Acidobacteria were detected was 1, and the number of persons in which a level of the Acidobacteria was equal to or lower than a detection limit was 23. In the HC group, the number of persons in which the Acidobacteria were detected was 16, and the number of persons in which the level of the Acidobacteria was equal to or lower than the detection limit was 5. When the FD group is compared with the HC group, the statistical significant difference was observed. As the p value at this time, p was smaller than 0.01 (p<0.01).

When a relationship between no detection of the Acidobacteria in the gastric juice of each of the subjects, and FD was examined, the sensitivity was 95.8% and the specificity was 76.2%. More specifically, the relationship between no detection of the Acidobacteria, and FD was shown.

### Example 2

A solid gastric flora improving agent containing Lactobacillus gasseri OLL2716 (FERM BP-6999) as an active ingredient was prepared according to the following method. Raw milk, powdered skim milk and water were used and appropriately prepared to be 3.0% by weight and 9.2% by weight in milkfat and non-fat milk solid, respectively. The resulting mixture was homogenized, sterilized and subjected to cooling treatment in the usual manner. Then, Lactobacillus bulgaricus, Streptococcus thermophilus and Lactobacillus gasseri OLL2716 (FERM BP-6999), which were separated from MEIJI PROBIO YOGURT LG21 of Meiji Co., Ltd,, were inoculated thereinto, and the mixture was cultured in the usual manner. The resulting culture was used for a test as a gastric flora improving agent. In addition, as this gastric flora improving agent, lactic acid bacteria containing the active ingredient are to be directly taken for convenience.

In this gastric flora improving agent, the number of the lactic acid bacteria belonging to Lactobacillus gasseri OLL2716 (FERM BP-6999) per gram was approximately 10⁷.

### (Test Method)

An intervention trial was conducted by using the gastric flora improving agent (hereinafter, referred to as a test sample in several cases).

Specifically, 24 patients having functional gastrointestinal disorders (a functional dyspepsia patient group, an FD group), who had no organic disease and were negative for H. pylori, were applied as targets. Moreover, as a control, 21 healthy persons (a healthy control group, an HC group) who had no organic disease and were negative for H. pylori were applied as targets.

Then, each of 24 patients in the FD group was allowed to take the gastric flora improving agent continuously for 12 weeks in a dose of 118 g per day.

Further, gastric juice of each of 24 patients in the FD group was directly collected from a stomach in a state of elapse of one night through a naso-gastric tube before intake of the gastric flora improving agent and after allowing each of the patients to continuously take the gastric flora improving agent for 12 weeks. Similarly, gastric juice of each of 21 persons in the HC group was also collected directly from the stomach in the state of elapse of one night through the naso-gastric tube.

With regard to identification of the flora in the gastric juice, the flora was analyzed by using a next-generation sequencer. An analysis method by using the next-generation sequencer was the same as in Example 1.

As a result, the Bacteroidetes occupancy relative to the Proteobacteria occupancy (Bacteroidetes occupancy/Proteobacteria occupancy) in the gastric juice in the FD group was reduced in 18 persons of 24 persons after administering the test sample (yogurt containing the lactic acid bacteria (Lactobacillus gasseri OLL2716 (FERM BP-6999)) for 12 weeks. Moreover, the number of patients in which a quotient (Bacteroides occupancy/Proteobacteria occupancy) obtained by dividing the Bacteroides occupancy by the Proteobacteria occupancy in the gastric juice in the FD group was more than 1 was 18 before administering the test sample for 12 weeks, but the number was reduced to 9 after administering the test sample for 12 weeks.

As described above, it was found that the patients having the functional gastrointestinal disorders are allowed to continuously take the test sample for 12 weeks, resulting in reduction of the Bacteroidetes occupancy relative to the Proteobacteria occupancy within the stomach, namely, the quotient (Bacteroides occupancy/Proteobacteria occupancy) obtained by dividing the Bacteroides occupancy by the Proteobacteria occupancy, and the functional gastrointestinal disorders can be improved.

Moreover, in the FD group, before the test sample was administered for 12 weeks, the number of persons in which the Acidobacteria were detected was 1, and the number of persons in which the level of the Acidobacteria was equal to or lower than the detection limit was 23, In the FD group, after the test sample was administered for 12 weeks, the Acidobacteria were detected at the occupancy of 0.1% or more from 24 persons being all the persons in the FD group.

As described above, it was found that the patients having the functional gastrointestinal disorders are allowed to continuously take the test sample for 12 weeks, resulting in a rise of the Acidobacteria occupancy within the stomach, and the functional gastrointestinal disorders can be improved.

1. A method of detecting presence or absence of a functional gastrointestinal disorder, comprising measuring bacteria occupancy within a human stomach.

2. The method according to claim 1, wherein the bacteria occupancy within the human stomach is measured on gastric juice collected from inside of the human stomach.

3. The method according to claim 1, comprising measuring that Bacteroidetes occupancy is higher than Proteobacteria occupancy within a human stomach and/or that Acidobacteria have no occupation within the human stomach.

4. The method according to claim 1, wherein the functional gastrointestinal disorder is functional dyspepsia.

5. The method according to claim 1, wherein the bacteria occupancy is measured by using a next-generation sequencer.

6.-4. (canceled)

15. A method of improving gastric flora, comprising, by administering a gastric flora improving agent containing lactic acid bacteria as an active ingredient, reducing Bacteroidetes occupancy relative to Proteobacteria occupancy within a stomach, and/or increasing Acidobacteria occupancy within the stomach.

16. The method according to claim 15, wherein the lactic acid bacteria are lactic acid bacteria of a genus Lactobacillus.

17. The method according to claim 15, wherein the lactic acid bacteria are Lactobacillus gasseri OLL2716 (FERM BP-6999).

18. The method according to claim 15, wherein a dose of the number of the lactic acid bacteria to a human per day is from 2×10⁷ to 5×10¹⁰.

19. The method according to claim 15, wherein, when a culture of the lactic acid bacteria contains 10⁷ or more of lactic acid bacteria per gram of the culture, a dose of the culture of the lactic acid bacteria to a human per day is 5 to 1000 g.

20. The method according to claim 15, wherein the gastric flora improving agent produces a gastric flora improving effect within 4 weeks after intake of the gastric flora improving agent.

21. The method according to claim 15, wherein the gastric flora improving agent is for use in persons negative for Helicobacter pylori and persons positive for Helicobacter pylori.

22. The method according to claim 15, wherein the gastric flora improving agent is for use in the persons negative for Helicobacter pylori.

23. The method according to claim 15, wherein the gastric flora improvement is measured by using a next-generation sequencer.
